(19) United States
(12) Patent Application Publication
(10) Pub. No.: US 2009/0215161 A1
(43) Pub. Date: Aug. 27, 2009

(54) MICROORGANISM TESTING DEVICE AND CHIP FOR TESTING MICROORGANISMS

(75) Inventors: Yasuhiko SASAKI, Tsuchiura (JP); Kei TAKENAKA, Kashiwa (JP); Tomoko SHINOMURA, Higashimurayama (JP); Akira MOCHIZUKI, Mito (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(73) Assignee: Hitachi, Ltd.

(21) Appl. No.: 12/360,183

(22) Filed: Jan. 27, 2009

(30) Foreign Application Priority Data
Feb. 21, 2008 (JP) 2008-039967

(51) Int. Cl. C12M 1/00

(52) U.S. Cl. 435/288.7; 435/287.1

(57) ABSTRACT

There is disclosed a microorganism testing device in which various types of microorganisms can be easily concentrated. The microorganism testing device includes a detection chip, a carrier device, a controller, and a magnet. The detection chip includes a specimen container for holding a specimen containing microorganisms, a trapping particle liquid container for holding trapping particle liquid containing magnetic particles, a microorganism trapping section for trapping the microorganisms, and a liquid flow path. In a state where a magnetic force of the magnet acts on the microorganism trapping section, the controller controls the carrier device to flow the trapping particle liquid into the microorganism trapping section to form a filtration filter by trapping plural magnetic particles in the microorganism trapping section, and in this state, to flow the microorganisms into the microorganism trapping section so that the microorganisms within the specimen are deposited on one side of the filtration filter.

### CLAIM OF PRIORITY

The present application claims priority from Japanese patent application JP 2008-039967 field on Feb. 21, 2008, the content of which is hereby incorporated by reference into this application.

### FIELD OF THE INVENTION

The present invention relates to a microorganism testing device and a chip for testing microorganisms. In particular, the present invention is suited for a microorganism testing device used for measurement of the number of living microorganisms, and a chip for testing microorganisms.

### BACKGROUND OF THE INVENTION

There have been known measurement devices for carrying out various types of simple and rapid measurement methods developed for speeding up and simplifying the measurement of the number of living microorganisms. In particular, the attention has been focused on a measurement device using fluorescent flow cytometry method as a method of rapidly and directly measuring the number of living microorganisms.

The fluorescence flow cytometry method is a particle measurement method that measures microorganisms by allowing them to flow one by one through a narrowed flow path through which a specimen containing the microorganisms stained with fluorescent dye flows. The measurement device using this method can measure the microorganisms one by one in a short period of time.

Further, in the fluorescence flow cytometry method, a laminar flow of a specimen and a sheath liquid is formed to narrow the flow diameter of the specimen by the pressure difference between the two liquids so that the microorganisms within the specimen liquid are prevented from being attached to the wall surface of the flow path.

Further, in order to reduce the price and the need of cleaning, there has been known a technology using a disposable chip at a flow path portion for the measurement performed by the fluorescence flow cytometry method. That is, the measurement is performed in this disposable chip, and the chip which is the flow path portion for the measurement is discarded after use. This technology is described, for example, in Journal of Biomolecular Techniques, Vol. 14, Issue 2, pp. 119-127.

### SUMMARY OF THE INVENTION

In the above described related art, rapid microorganism measurement of a large volume specimen is not taken into account. In order to measure the number of living microorganisms contained in the specimen, an inspector has to concentrate the specimen before injecting the specimen into a well. Such operations require the inspector to have a specialized skill to prevent reduction in the number of living microorganisms.

It is an object of the present invention to provide a microorganism testing device capable of easily concentrating various types of microorganisms, and a chip for testing microorganisms.

In order to achieve the above object, a first aspect of the present invention includes: a detection chip having therein a specimen container for holding a specimen containing microorganisms, a trapping particle liquid container for holding a trapping particle liquid containing magnetic particles, a microorganism trapping section for trapping the microorganisms contained in the specimen, and a liquid flow path; a carrier device for applying a carrying force to the specimen held in the specimen container and to the trapping particle liquid held in the trapping particle liquid container; a controller for controlling the carrier device; a magnet for holding the magnetic particles in the microorganism trapping section by a magnetic force; and a detector for detecting the microorganisms flowing in the detection chip. In a state where the magnetic force of the magnet acts on the microorganism trapping section, the controller controls the carrier device to flow the trapping particle liquid into the microorganism trapping section to form a filtration filter by trapping and holding the plural magnetic particles in the microorganism trapping section. In this state, the controller controls the carrier device to flow the specimen into the microorganism trapping section so that the microorganisms within the specimen are deposited on one side of the filtration filter.

A more preferred example in the first aspect of the present invention will be described below.
(1) The detection chip has therein a removing liquid container for holding a removing liquid. The carrier device applies a carrying force to the removing liquid held in the removing liquid container. The microorganism trapping section has a magnetic particle holding filter provided in a portion of the liquid flow path, to form the filtration filter by trapping the magnetic particles on the magnetic particle holding filter.
(2) In the above described (1), in a process for removing the microorganisms deposited in the filtration filter, the magnet changes from a first state where the magnetic particle holding force for holding the magnetic particles is larger than the magnetic particle removing force for removing the magnetic particles from the magnetic particle holding filter, to a second state where the magnetic particle holding force for holding the magnetic particles is smaller than the magnetic particle removing force for removing the magnetic particles from the microorganism trapping section.
(3) In the above described (2), the magnetic particle holding force of the magnet for holding the magnetic particles is gradually reduced to allow the magnetic particles to gradually flow out of the magnetic particle holding filter.
(4) In the above described (3), the magnet is movably provided on the side opposite to the filtration filter with the magnetic particle holding filter interposed therebetween.
(5) In the above described (1), the removing liquid is allowed to flow, in the initial stage of a process for removing the microorganisms, by applying a magnetic force to hold all the magnetic particles on the magnetic particle holding filter. In the intermediate stage of the process for removing the microorganisms, the removing liquid is allowed to flow by reducing the magnetic force to a level to hold some of the magnetic particles on the magnetic particle holding filter. In the late stage of the process for removing the microorganisms, the removing liquid is allowed to flow by further reducing the magnetic force to a level to allow all the magnetic particles to flow out of the magnetic particle holding filter.
(6) In the above described (1), the detection chip has a multi-layer structure including a front member, an intermediate member, and a rear member. The microorganism trapping section has grooves formed on both surfaces of the intermediate member, a through hole through which the grooves communicate with each other, and the magnetic particle holding filter provided in the through hold.
(7) In the above described (1), the volume of the removing liquid held in the removing liquid container is smaller than the volume of the specimen container.
(8) In the above describe (1), the detection chip has therein a staining reagent container and a detection section. The carrier device applies a carrying force to the staining reagent held in the staining reagent container. The controller controls the carrier device to flow the staining reagent into the microorganism trapping section to stain the microorganisms deposited in the filtration filter, allowing a detection liquid that contains the removing liquid as well as the microorganisms stained and removed from the filtration filter, to flow into the detection section. The detector irradiates light onto the detection liquid flowing through the detection section, detects fluorescence or scattering light from the stained microorganism, and converts the detected light into electrical signals to measure the number of the relevant microorganisms.

A second aspect of the present invention includes: a specimen container for holding a specimen; a residue removing section for removing residues within the specimen; a microorganism trapping section for trapping microorganisms within the specimen; a trapping particle liquid container for holding magnetic particles to form a filtration filter in the microorganism trapping section; a staining reagent container for holding staining reagent; a filtered liquid waste container for receiving the specimen, the trapping particle liquid, and the staining reagent that have passed through the microorganism trapping section; a removing liquid container for holding a removing liquid; a detection liquid container for holding a detection liquid which is a liquid of the microorganisms removed from the microorganism trapping section; a detection section for detecting the microorganisms; a detection liquid waste container for receiving the detection liquid having passed through the detection section; a liquid flow path for connecting the respective containers, through which the specimen, the trapping particle liquid, the staining reagent, and the removing liquid flow; a vent for allowing the specimen, the trapping particle liquid, the staining reagent, and the removing liquid to flow by air pressure; and an air flow path for connecting the vent and the respective containers. The microorganism trapping section includes a magnetic particle holding filter to form a filtration filter by depositing the plural magnetic particles, in which a magnetic force is applied to the magnetic particles forming the filtration filter.

According to the present invention, it is possible to provide a microorganism testing device capable of easily concentrating various types of microorganisms, and a chip for testing microorganisms.

### BRIEF DESCRIPTION OF THE DRAWINGS

FIG. 1 is an overall view of a microorganism testing device according to an embodiment of the present invention;

FIG. 2 is a system block diagram of the microorganism testing device of FIG. 1;

FIG. 3 is a front view of a detection chip of FIG. 1;

FIG. 4 is a vertical cross-sectional view showing a part of the detection chip of FIG. 1;

FIG. 5 is an enlarged front view of a detection section of the detection chip of FIG. 1;

FIG. 6 is a vertical cross-sectional view of the detection section of FIG. 5;

FIG. 7 is a block diagram of an optical system of a detector of FIG. 1;

FIG. 8 is a process diagram of a microorganism measurement performed in the detection chip of FIG. 1;

FIG. 9 is a diagram showing the flow of the trapping particle liquid in the detection chip of FIG. 1;

FIG. 10 is a vertical cross-section view of a microorganism trapping section in the flow of the trapping particle liquid in FIG. 9;

FIG. 11 is a diagram showing the flow of a specimen liquid in the detection chip of FIG. 1;

FIG. 12A is a vertical cross-sectional view of a microorganism trapping section in the initial stage of the flow of the specimen liquid in FIG. 11;

FIG. 12B is a vertical cross-sectional view of the microorganism trapping section in the late stage of the flow of the specimen liquid in FIG. 11;

FIG. 13 is a diagram showing the flow of staining reagent in the detection chip of FIG. 1;

FIG. 14 is a diagram showing the flow of a removing liquid in the detection chip of FIG. 1;

FIG. 15 is a vertical cross-sectional view of the microorganism trapping section in the initial stage of the flow of the detection/removing liquid in FIG. 14;

FIG. 16 is a vertical cross-sectional view of the microorganism trapping section in the intermediate stage of the flow of the detection/removing liquid in FIG. 14;

FIG. 17 is a vertical cross-sectional view of the microorganism trapping section in the late stage of the flow of the detection/removing liquid in FIG. 14;

FIG. 18 is a diagram showing the changes in the magnetic particle holding force and the magnetic particle removing force according to the present embodiment; and

FIG. 19 is a diagram showing the flow of the detection liquid in the detection chip of FIG. 1.

### DETAILED DESCRIPTION OF THE PREFERRED EMBODIMENT

Hereinafter, a microorganism testing device according to an embodiment of the present invention will be described with reference to the accompanying drawings.

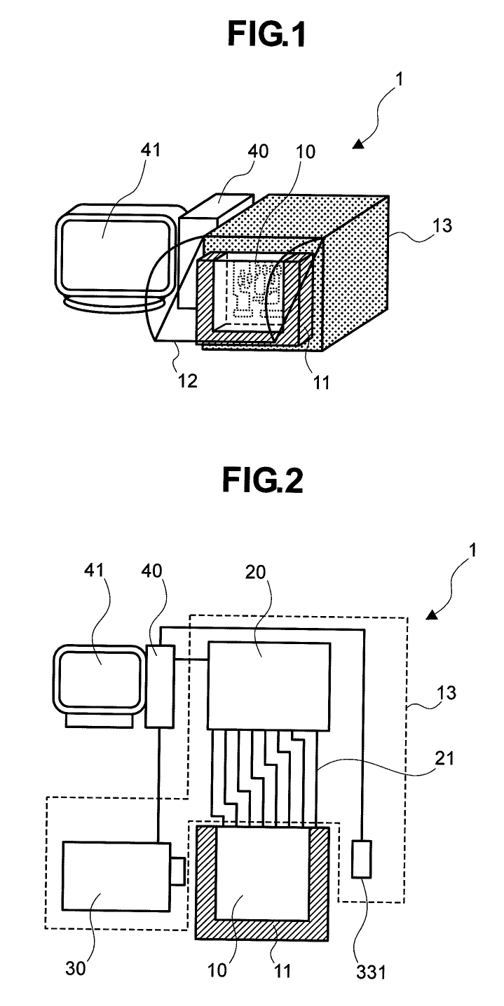

First, a general outline of a microorganism testing device 1 of this embodiment will be described with reference to FIGS. 1 and 2. FIG. 1 is an overall view of the microorganism testing device 1 of this embodiment. FIG. 2 is a system block diagram of the microorganism testing device 1 of FIG. 1.

The microorganism testing device 1 includes a detection chip 10, a holder 11, a cover 12, a detector body 13, a controller 40 for controlling each component of the detector body 13, and an output device 41 connected to the controller 40.

The detection chip 10 is configured as a single disposable chip holding therein a specimen and staining reagent, and having therein a mechanism to perform processing necessary for measuring microorganisms. The detection chip 10 is attached to the holder 11 when used. The detection chip 10 is held in front of the detector body 13 by the holder 11 and the cover 12. The microorganism measurement process can be made suitable for various types of specimens by changing the type of the detection chip 10 to be attached.

The holder 11 has a function of controlling the temperature of the detection chip 10, in addition to holding the detection chip 10. The cover 12 is formed of a transparent material, and covers the detection chip 10.

The detector body 13 includes a carrier device 20 for carrying the liquid in the detection chip 10, a detector 30 for detecting microorganisms flowing in the detection chip 10, and a magnet 331 for applying a magnetic force to magnetic particles in the detection chip 10.

The carrier device 20 is connected to the detection chip 10 through chip connecting tubes 21. The carrier device 20 carries the specimen, staining reagent, removing liquid, and the like, which are held in the detection chip 10, to perform processing necessary for measuring microorganisms.

The detector 30 irradiates excitation light onto each microorganism flowing in the detection chip 10. Then, the detector 30 detects fluorescence from the irradiated microorganism, converts the detection result into an electrical signal, and transmits the signal to the controller 40. In this embodiment, the detector 30 detects the microorganisms by the fluorescent flow cytometry method.

The controller 40 performs control of each component of the detector body 13, and processing of electrical signals output from each component, and the like. Then, the controller 40 outputs the obtained detection result to the output device 41. The output device 41 displays the detection result on a screen. Incidentally, the output device 41 may include a printer so that the detection result can be printed out on paper.

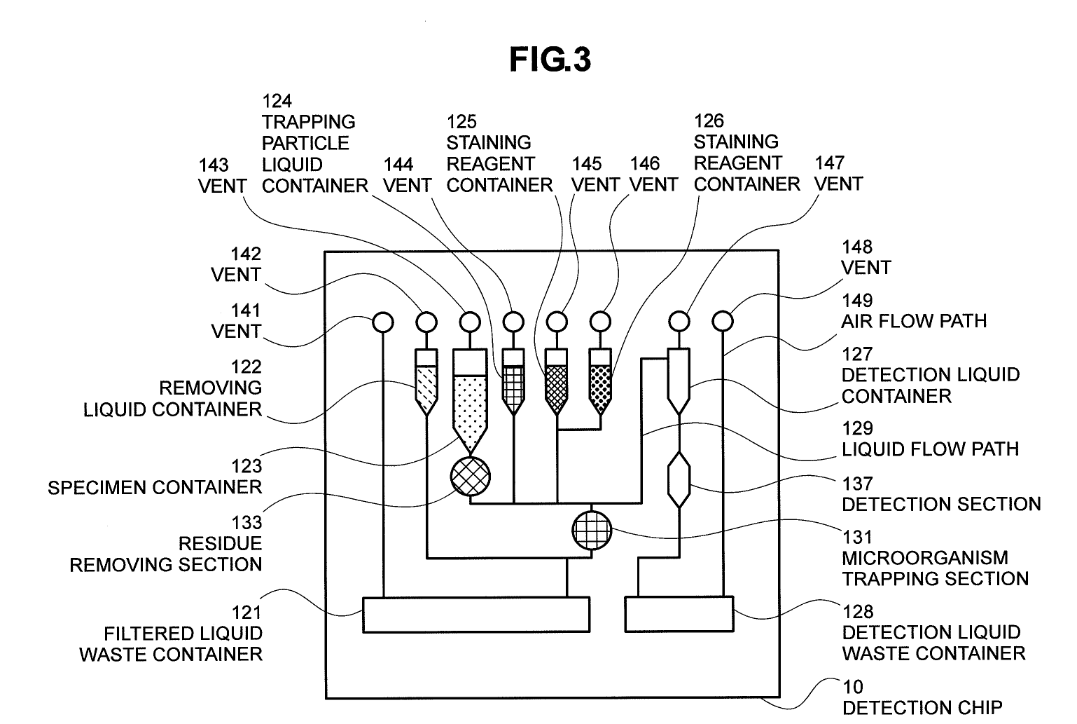

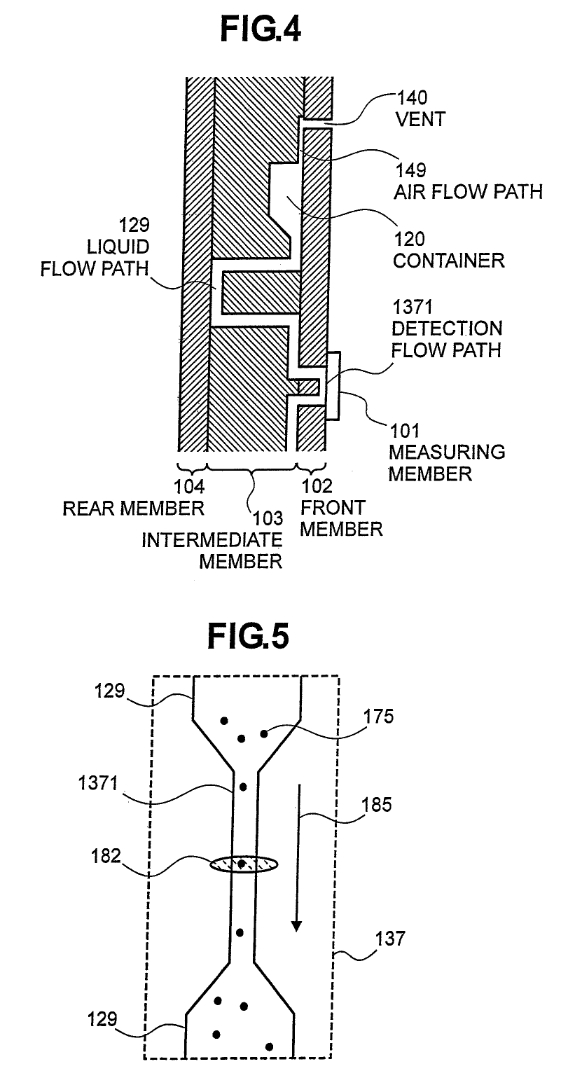

Next, the detection chip 10 will be described in detail with reference to FIGS. 3 and 4. FIG. 3 is a front view of the detection chip 10 of FIG. 1. FIG. 4 is a vertical cross-sectional view showing a part of the detection chip 10 of FIG. 1.

As shown in FIG. 3, the detection chip 10 includes: a specimen container 123 for holding a specimen, a residue removing section 133 for removing residues within the specimen; a microorganism trapping section 131 for trapping microorganisms within the specimen; a trapping particle liquid container 124 for holding the trapping particles to form a filtration filter in the microorganism trapping section 131; staining reagent containers 125, 126 for holding staining reagents; a filtered liquid waste container 121 for receiving the specimen, the trapping particle liquid, and the staining reagents that have passed through the microorganism trapping section 131; a removing liquid container 122 for holding a removing liquid; a detection liquid container 127 for holding a liquid of the microorganisms removed from the microorganism trapping section, namely, a detection liquid; a detection section 137 for detecting the microorganisms; a detection liquid waste container 128 for receiving the detection liquid having passed through the detection section 137; a liquid flow path 129 for connecting the respective containers 121 to 128, through which the specimen, the trapping particle liquid, the staining reagents, and the removing liquid flow; vents 141 to 148 for allowing the specimen, the trapping particle liquid, the staining reagents, and the removing liquid to flow by the air pressure; and an air flow path 149 for connecting the vents 141 to 148 and the containers 121 to 128, respectively.

The plural containers 122 to 127 are disposed in an upper portion of the detection chip 10. Each of the containers 122 to 127 is formed to extend in the longitudinal direction. The containers 121 to 128 are also collectively referred to as container 120.

The plural vents 141 to 148 are disposed in the upper portion of the detection chip 10, which are located above the containers 121 to 128. The vents 141 to 148 are also collectively referred to as vent 140.

The liquid flow path 129 has a depth and width in the range of 10 μm to 1 mm. Similarly, the air flow path 149 has a depth and width in the range of 10 μm to 1 mm. In consideration of the delivery of liquids, the cross sectional area of the liquid flow path 129 is made larger than the cross sectional area of the air flow path 149.

The staining reagents are previously included in the staining reagent containers 125, 126. This reduces, to a minimum, the influence of degradation due to the outside environment as well as the possibility that an inspector could touch the reagents. The specimen is injected into the specimen container 123 from the vent 143 before testing.

The volume of the specimen container 123 is larger than the volume of the specimen. Further, the highest point of the liquid flow path 129, which connects the microorganism trapping section 131 and the detection liquid container 127, is made higher than the water level of the detection liquid within the detection liquid container 127.

Examples of the staining reagent include dyes for staining microorganisms, such as DAPI (1 μg/ml to 1 mg/ml), acridine orange (1 μg/ml to 1 mg/ml), and ethidium bromide (1 μg/ml to 1 mg/ml).

As shown in FIG. 4, the detection chip 10 has a four-layer structure including a measuring member 101, a front member 102, an intermediate member 103, and a rear member 104. The measuring member 101 is formed using optical transparent materials such as glass, quartz, polymethacrylic acid ester, and PDMS. The front member 102, the intermediate member 103, and the rear member 104 are formed using materials such as polymethacrylic acid ester, ABS, polycarbonate, and PDM that have undergone a light-shielding process to prevent degradation of the staining reagents by the outside light.

The intermediate member 103 has grooves on the surfaces to which the front member 102 and the rear member 104 are attached. The front member 102, the rear member 104, and the intermediate member 103 are attached to each other. At this time, deep grooves form the container 120 for holding the specimen and the reagents. Shallow grooves form the liquid flow path 129 through which the specimen and the reagents flow, as well as the air flow path 149 through which the air flows. The grooves formed on the both surfaces of the intermediate member 103 are connected with through holes. In this way, a flow path is formed by the grooves and the through holes.

The front member 102 has a groove on a surface contacting with the measuring member 101, and through holes for connecting the groove of the front member 102 and the groove of the intermediate member 103. The measuring member 101 and the front member 102 are attached to each other to form a detection flow path 1371 allowing optical measurement from the outside. Fluorescence from fluorescently-stained microorganisms can be measured through the measuring member 101. The through holes form the vent 140, and a flow path for connecting the detection flow path 1371 and the liquid flow path 129.

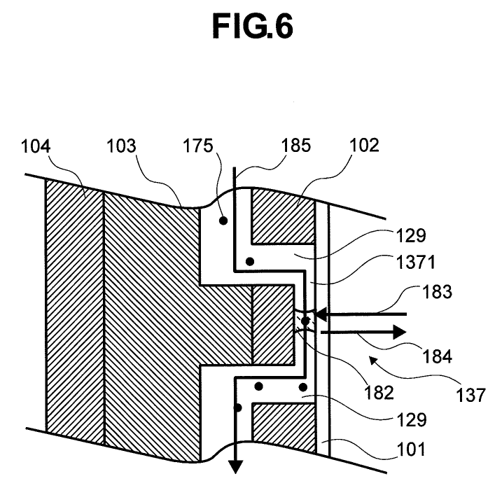

Next, the detection section 137 of the detection chip 10 will be described in detail with reference to FIGS. 5 and 6. FIG. 5 is an enlarged front view of the detection section 137 of the detection chip 10. FIG. 6 is a vertical cross-sectional view of the detection section 137 of the detection chip 10. The measurement of microorganisms 175 in the detection section 137 is performed by the detection device 30, using the fluorescent flow cytometry method.

In the detection section 137, the detection flow path 1371 is designed to have a width and depth in the range of 1 μm to 0.1 mm, and a length in the range of 10 μm to 10 mm, respectively. In addition, the length of the flow path is made longer than the width and depth thereof. The cross-sectional area of the detection flow path 1371 is made smaller than the cross-sectional areas of the liquid flow path 129 before and after the detection flow path 1371. Since the detection flow path 1371 is a very narrow flow path, it rarely occurs that two or more microorganisms 175 flow side by side therethrough. In other words, the detection flow path 1371 is designed to allow the microorganisms 175 to flow one by one.

Upon detection of the microorganisms 175, an excitation light 183 from the detector 30 is injected through the measuring member 101 into the detection flow path 1371. The excitation light 183 is concentrated and output in the form of an ellipse in the detector 30. The incident area of the excitation light 183 to the detection section 137 is concentrated to an irradiation area 182. The stained microorganism 175 flows in the direction of an arrow 185, and emits fluorescence 184 when the microorganism 175 passes through the irradiation area 182. The fluorescence 184 is detected in the detector 30 through the measuring member 101.

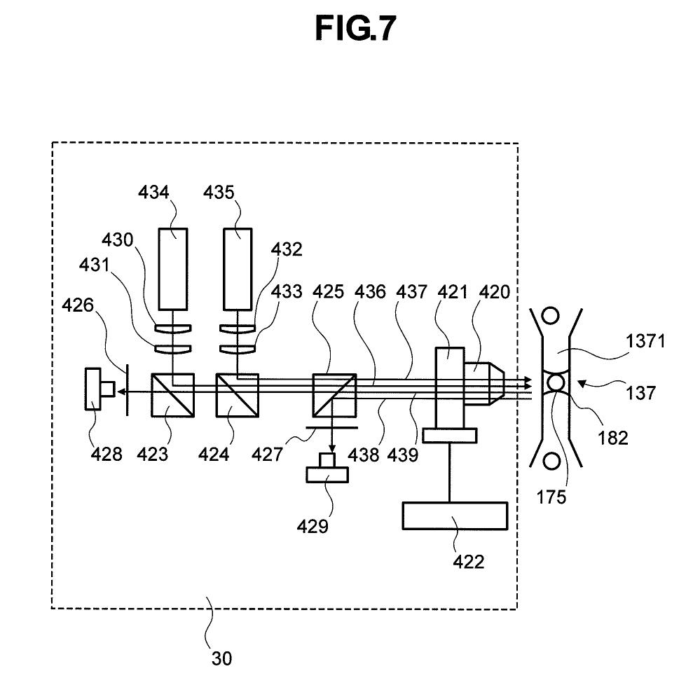

Next, the detector 30 will be described in detail with reference to FIG. 7. FIG. 7 is a block diagram of the optical system of the detector 30 of FIG. 1. The optics and their placement may differ depending on the excitation spectrum and fluorescence spectrum of the staining dye to be used. Here, description will be made on the optical system supporting two types of staining dyes, ethidium bromide (excitation wavelength of 520 nm, fluorescence wavelength of 615 nm) and DAPI (excitation wavelength of 360 nm, fluorescence wavelength of 460 nm).

The detector 30 includes: a short wavelength laser 434 (wavelength of 360 nm) as a source of excitation light of short wavelength (for DAPI); a long wavelength laser 435 (wavelength of 520 nm) as a source of excitation light of long wavelength (for ethidium bromide); cylindrical lenses 430 to 433 for concentrating the laser light from the lasers 434, 435 in the form of an ellipse; a short-wavelength dichroic mirror 423 for reflecting the light having a wavelength of 400 nm or less; an intermediate-wavelength dichroic mirror 424 for reflecting the light having a wavelength of 500 nm or more; a long-wavelength dichroic mirror 425 for reflecting the light having a wavelength of 600 nm or more; a short-wavelength optical filter 426 not allowing the light having a wavelength of 500 nm or more to pass through; a long-wavelength optical filter 427 not allowing the light having a wavelength of 700 nm or more to pass through; a short wavelength photomultiplier 428 for detecting the light passing through the short-wavelength optical filter 426; a long wavelength photomultiplier 429 for detecting the light passing through the long-wavelength optical filter 427; an objective lens 420 for concentrating the fluorescence from the microorganism 175; a piezo 421 for moving the objective lens 420 at a high speed; and a piezo controller 422 for controlling the movement of the piezo.

The excitation light 436 (wavelength of 360 nm) output from the short wavelength laser 434 is concentrated in the form of an ellipse by the cylindrical lenses 430, 431. Then, the light 436 is reflected by the short-wavelength dichroic mirror 423, and is irradiated onto the irradiation area 182 through the intermediate-wavelength dichroic mirror 424, the long-wavelength dichroic mirror 425, and the objective lens 420. This excites DAPI with which the microorganism 175 flowing through the irradiation area 182 is stained. Fluorescence 439 (wavelength of 460 nm) from the DAPI is injected into the short wavelength photomultiplier 428, through the long-wavelength dichroic mirror 425, the intermediate-wavelength dichroic mirror 424, the short-wavelength dichroic mirror 423, and the short-wavelength optical filter 426. The fluorescence 439 detected by the short wavelength photomultiplier 428 is converted into an electrical signal. Then, the electrical signal is transmitted to the controller 40.

While an excitation light 437 (wavelength of 530 nm) output from the long wavelength laser 435 is concentrated in the form of an ellipse by the cylindrical lenses 423, 433. Then, the light is reflected by the intermediate-wavelength dichroic mirror 424, and is irradiated onto the irradiation area 482 through the long-wavelength dichroic mirror 425 and the objective lens 420. This excites ethidium bromide with which the microorganism 175 flowing through the irradiation area 482 is stained. Fluorescence 438 (wavelength of 620 nm) from the ethidium bromide is reflected by the long-wavelength dichroic mirror 425, and is injected into the long wavelength photomultiplier 429 through the long-wavelength optical filter 427. The fluorescence 438 detected by the long wavelength photomultiplier 429 is converted into an electrical signal. Then, the electrical signal is transmitted to the controller 40.

The controller 40 processes the electrical signals transmitted from the short wavelength photomultiplier 428 and from the long wavelength photomultiplier 429. The controller 40 outputs the information of the number of microorganisms as the detection result, to the output device 41. The output device 41 displays the detection result.

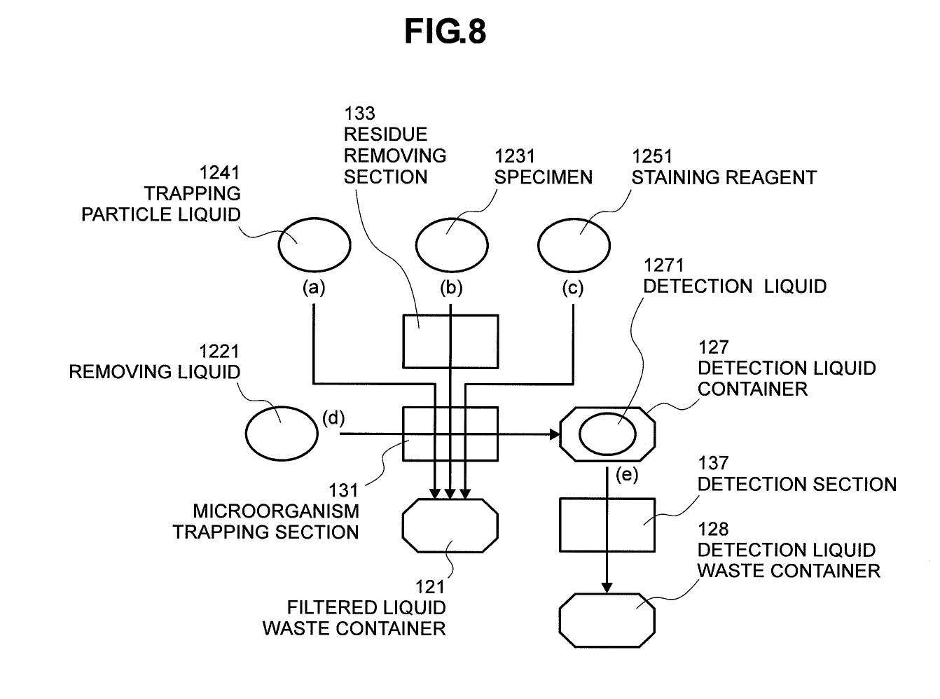

Next, an outline of microorganism measurement will be described with reference to FIG. 8. FIG. 8 is a process diagram of a microorganism measurement performed in the detection chip of FIG. 1. In the figure, reference symbols (a) to (e) denote process routes of a trapping particle liquid 1241, specimen 1231, staining reagent 1251, removing liquid 1221, and detection liquid 1271, respectively.

As described above, the detection chip 10 includes the residue removing section 133 for removing residues larger than the microorganisms 175 from the specimen 1231, the microorganism trapping section 131 for trapping and concentrating the microorganisms 175 within the specimen 1231, and the detection section 137 for detecting the microorganisms 175.

An outline of processes for measuring the microorganisms 175 will be described in accordance with the process routes (a) to (e). The processes are switched and performed by the controller 40 controlling the carrier device 20.

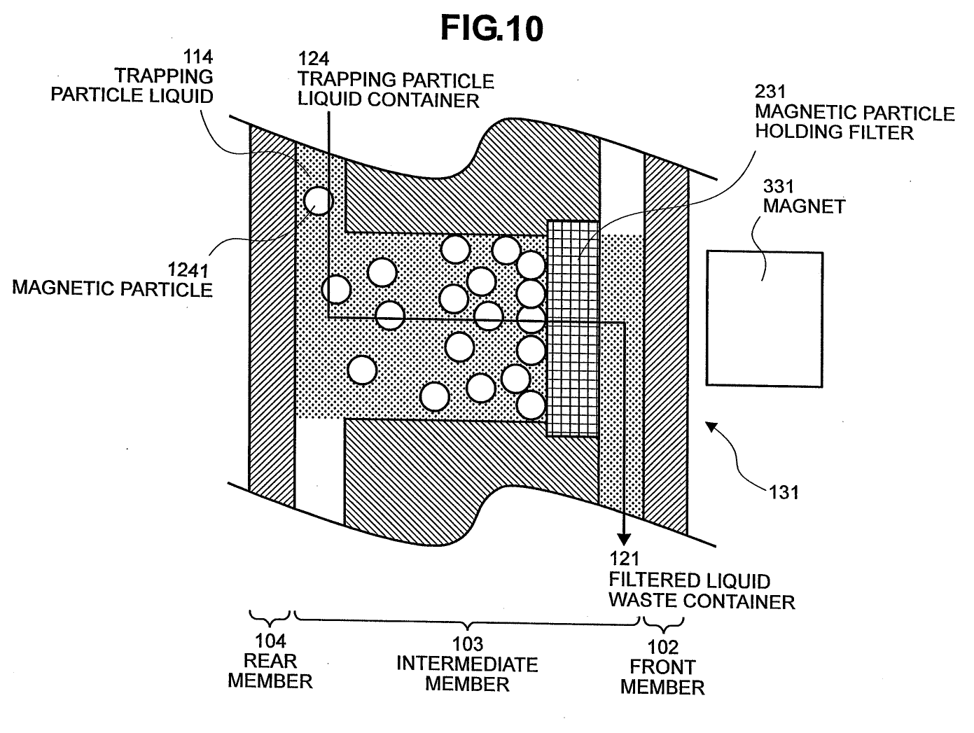

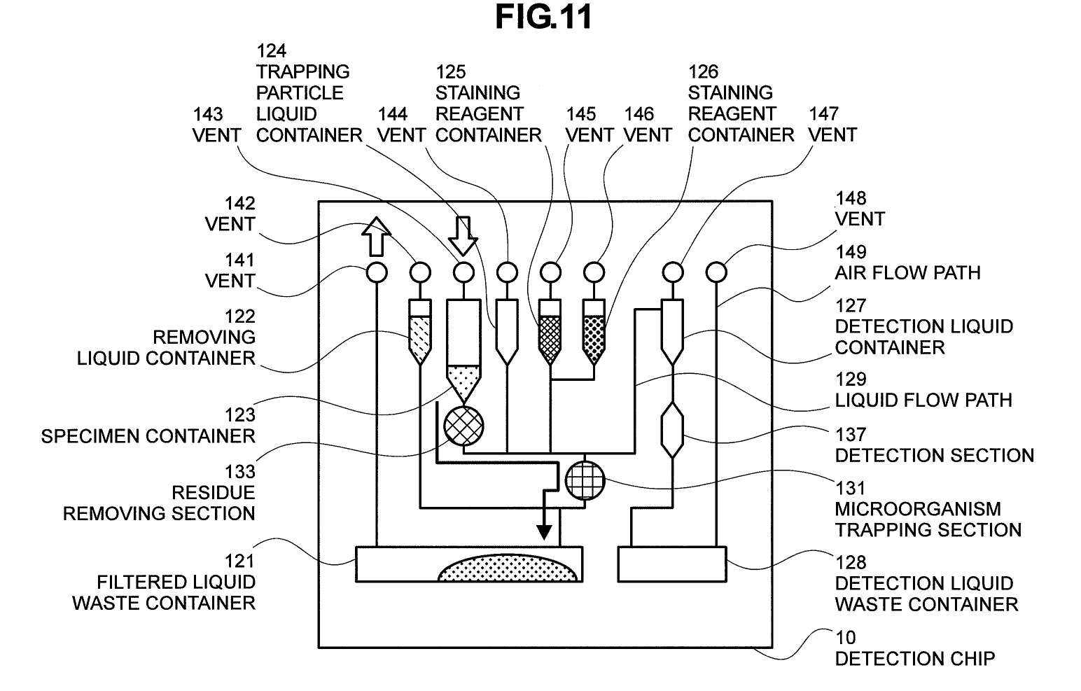

First, according to the process route indicated by (a), a process for forming a filtration filter is performed. The trapping particle liquid 1241 is pushed out of the trapping particle liquid container 124 by an operation of the carrier device 20. Then, the trapping particle liquid 1241 passes through the microorganism trapping section 131 into the filtered liquid waste container 121, and is removed. At the time of passing through the microorganism trapping section 131, the trapping particles (magnetic particles 214 shown in FIG. 10) within the trapping particle liquid 1241 are deposited in the microorganism trapping section 131. The filtration filter is formed with the magnetic particles 214.

Next, according to the process route indicated by (b), a process for removing residues within the specimen and a process for trapping microorganisms are performed. The specimen 1231 is pushed out of the specimen container 123 by an operation of the carrier device 20. The specimen 1231 passes through the residue removing section 133 and the microorganism trapping section 131. At this time, residues larger than the microorganisms 175 within the specimen 1231 are removed in the residue removing section 133. Then, the microorganisms 175 within the specimen 1231 are trapped in the microorganism trapping section 131. Incidentally, residues such as the dyes smaller than the microorganisms 175 pass through the microorganism trapping section 131 into the filtered liquid waste container 121, together with the specimen. Then, the residues are removed in the filtered liquid waste container 121.

Next, according to the process route indicated by (c), a process for staining the microorganisms 175 is performed. The staining reagent 1251 for staining the microorganisms 175 is pushed out of the staining reagent container 125 or 126 by an operation of the carrier device 20, and passes through the microorganism trapping section 131. At this time, the staining reagent 1251 stains the microorganisms 175 trapped in the microorganism trapping section 131. The excess of the staining reagent 1251 that have passed through the microorganism trapping section 131 enters the filtered liquid waste container 121, and is removed.

Next, according to the process route indicated by (d), a process for removing the microorganisms 175 stained with the fluorescent dye is performed. The removing liquid 1221 for removing the microorganisms 175 trapped in the microorganism trapping section 131, is pushed out of the removing liquid container 122 by an operation of the carrier device 20, and passes through the microorganism trapping section 131. At this time, the removing liquid 1221 removes the microorganisms 175 from the microorganism trapping section 131, and enters the detection liquid container 127 together with the microorganisms 175. Thus, the detection liquid 1271 is given.

Next, according to the process route indicated by (e), a process for detecting the microorganisms 175 stained with the fluorescent dye is performed. The detection liquid 1271 enters the microorganism detection section 137 from the detection liquid container 127. The microorganisms 175 within the detection liquid 1271 are measured in the microorganism detection section 137. After completion of the measurement in the microorganism detection section 137, the detection liquid 1271 enters the detection liquid waste container 128, and is removed.

The above processes are all performed in the detection chip 10. This reduces the possibility that the inspector will touch the microorganisms 175 within the specimen 1231 as well as the staining reagent 1521, thus reducing the influence on the detection result due to an error made by the inspector or outside influences.

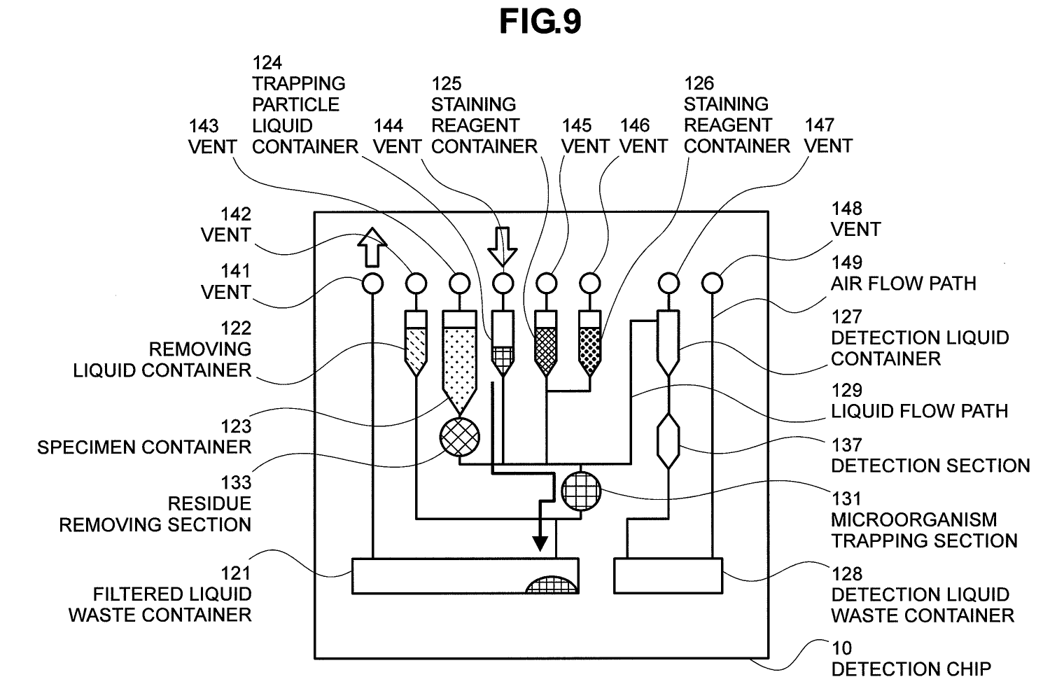

Next, the process for forming the filtration filter in the microorganism measurement will be described in detail with reference to FIGS. 9 and 10. FIG. 9 is a diagram showing the flow of the trapping particle liquid 1241 in the detection chip 10 of FIG. 1. FIG. 10 shows a vertical cross-sectional view of the microorganism trapping section 131 in the flow of the trapping particle liquid 1241 in FIG. 9.

As shown in FIG. 9, the air pressure within the trapping particle liquid container 124 is raised by applying the pressure from the carrier device 20 to the trapping particle liquid container 124 through the vent 144. At the same time, the filtered liquid waste container 121 is opened to the atmosphere through the vent 141. The other vents 142, 143, and 145 to 148 are closed. The trapping particle liquid 1241 flows from the particle liquid container 124 to the filtered liquid waste container 121 through the microorganism trapping section 131, due to the difference between the air pressures in the two containers 124 and 121. When the trapping particle liquid 1241 passes through the microorganism trapping section 131, as shown in FIG. 10, the magnetic particles 214 within the trapping particle liquid 1241 are deposited in the microorganism trapping section 131. Thus, the filtration filter is formed.

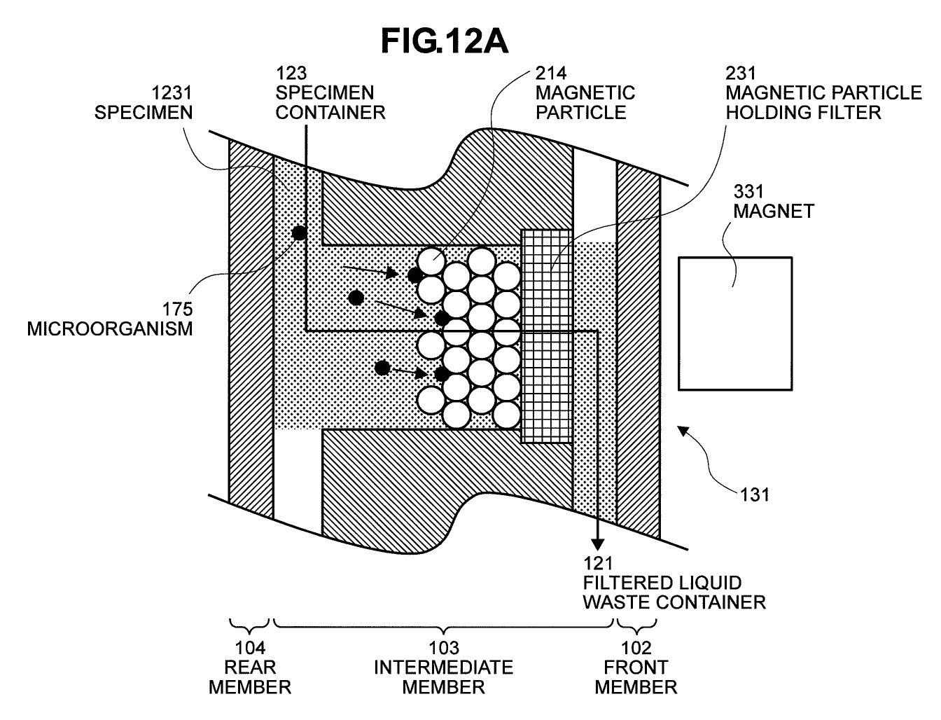

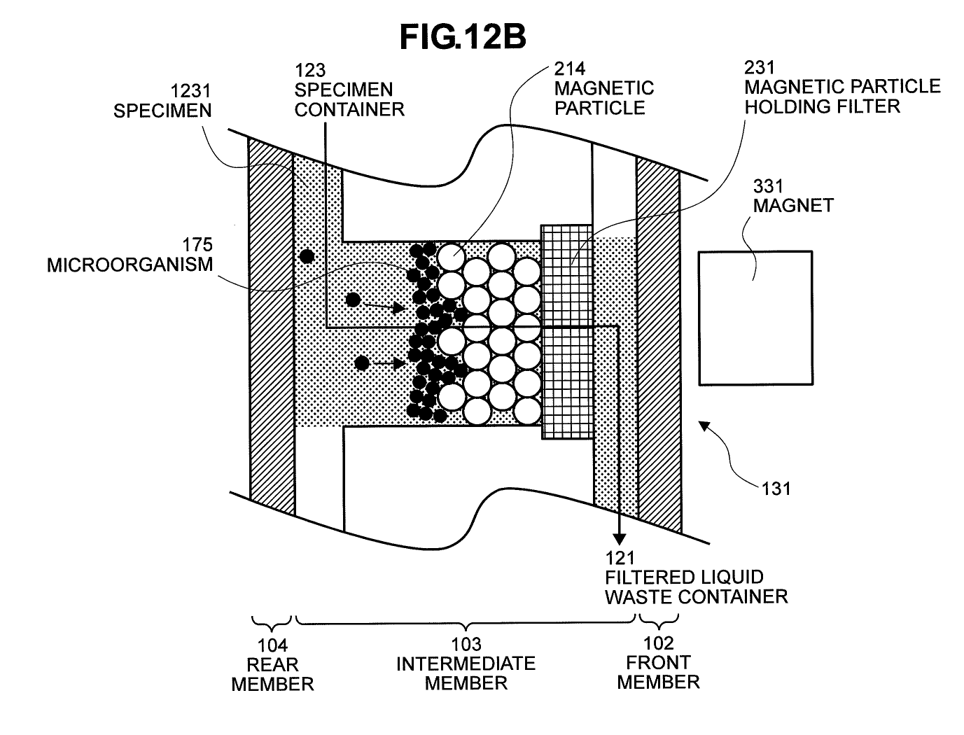

As shown in FIG. 10, the microorganism trapping section 131 has a magnetic particle holding filter 231 provided in the through hole of the intermediate member 103. The pore diameter of the magnetic particle holding filter 231 is made smaller than the diameter of the magnetic particles 214. For this reason, when the trapping particle liquid 124 is caused to pass through the magnetic particle holding filter 231, as shown in FIG. 10, the magnetic particles 214 within the trapping particle liquid 1241 are sequentially deposited on one side of the magnetic particle holding filter 231. Finally, as shown in FIG. 12, the magnetic particles 214 are deposited to form a filtration filter. In this way, it is possible to easily accumulate the magnetic particles 214 by providing the magnetic particle holding filter 231 on one side of the through hole.

In this embodiment, the size of the magnetic particles 214 is made about twice larger than the size of the microorganisms 175 to be trapped. The pore diameter (the dimension of the gap formed between the magnetic particles 214) as the filtration filter formed with the magnetic particles 214, is smaller than (namely, less than half) the outer diameter of the microorganisms 175. The pore diameter is small enough to trap the microorganisms 175.

Further, when the trapping particle liquid 1241 is caused to pass through the magnetic particle holding filter 231, a magnet 331 is placed in the vicinity of the magnetic particle holding filter 231. The magnet 331 is placed on the opposite side to the position in which the magnetic particles 124 are deposited, with the magnetic particle holding filter 231 interposed therebetween. The magnetic force of this magnet 331 can continuously attract the magnetic particles 214 to the magnetic particle holding filter 231. Thus, it is possible to prevent the filter structure formed with the magnetic particles 214, from being collapsed by the flow of the specimen. In addition, it is also possible to deposit the magnetic particles 214 without being affected by gravity. This facilitates the installation of the microorganism trapping section 131 in the detection chip 10.

Next, the process for trapping the microorganisms within the specimen 1231 will be described in detail with reference to FIG. 11 and FIGS. 12A, 12B. FIG. 11 is a diagram showing the flow of the specimen 1231 in the detection chip 10 of FIG. 1. FIG. 12A is a vertical cross-sectional view of the microorganism trapping section 131 in the initial stage of the flow of the specimen 1231 in FIG. 11. FIG. 12B is a vertical cross-sectional view of the microorganism trapping section 131 in the late stage of the flow of the specimen 1231 in FIG. 11.

As shown in FIG. 11, the air pressure within the specimen container 123 is raised by applying the pressure from the carrier device 20 to the specimen container 123 through the vent 143. At the same time, the filtered liquid waste container 121 is opened to the atmosphere through the vent 141. The other vents 142 and 144 to 148 are closed. The specimen 1231 flows from the specimen container 123 to the filtered liquid waste container 121 through the microorganism trapping section 131, due to the difference between the air pressures in the two containers. When the specimen 1231 passes through the microorganism trapping section 131, as shown in FIGS. 12A, 12B, the microorganisms 175 are trapped by the filtration filter formed in the microorganism trapping section 131. In this way, the microorganisms 175 are deposited and then concentrated.

In other words, since the size of the microorganisms 175 is larger than the pore diameter as the filtration filter formed with the magnetic particles 214, the microorganisms 175 are first deposited corresponding to the holes of the filtration filter as shown in FIG. 12A. Then, the microorganisms 175 are further deposited on the deposited microorganisms 175 as shown in FIG. 12B. This makes it possible to deposit a large amount of the microorganisms, even if the microorganisms 175 are not attracted by the magnetic particles 214. In other words, it is possible to deposit various types of the microorganisms 175, regardless of the characteristics of the microorganisms 175.

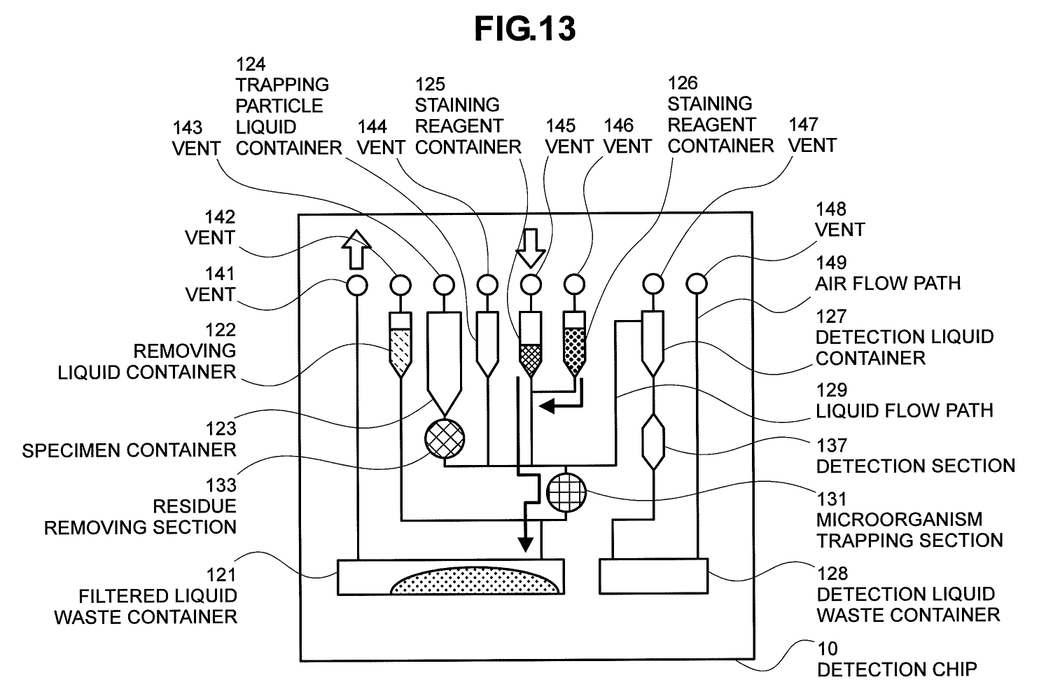

Next, the process for staining the microorganisms 175 will be described in detail with reference to FIG. 13. FIG. 13 is a diagram showing the flow of the staining reagents in the detection chip 10 of FIG. 1.

The air pressure within the staining reagent container 125 is raised by applying the pressure from the carrier device 20 to the staining reagent container 125 through the vent 145. At the same time, the filtered liquid waste container 121 is opened to the atmosphere through the vent 141. The other vents 142 to 144 and 146 to 148 are closed. The staining reagent flows from the staining reagent container 125 to the filtered liquid waste container 121 through the microorganism trapping section 131, due to the difference between the air pressures in the two containers 125 and 121. At the time of passing through the microorganism trapping section 131, the staining reagent stains the microorganisms 175 trapped by the filtration filter of the trapping particles 214 that is formed in the microorganism trapping section 131.

Similarly, the air pressure within the staining reagent container 126 is raised by applying the pressure from the carrier device 20 to the staining reagent container 126 through the vent 146. At the same time, the filtered liquid waste container 121 is opened to the atmosphere through the vent 141. The other vents 142 to 145, 147 and 148 are closed. The staining reagent flows from the staining reagent container 126 to the filtered liquid waste container 121 through the microorganism trapping section 131, due to the difference between the air pressures in the two containers 126 and 121. At the time of passing through the microorganism trapping section 131, the staining regent stains the microorganisms 175 trapped by the filtration filter of the trapping particles 214 that is formed in the microorganism trapping section 131.

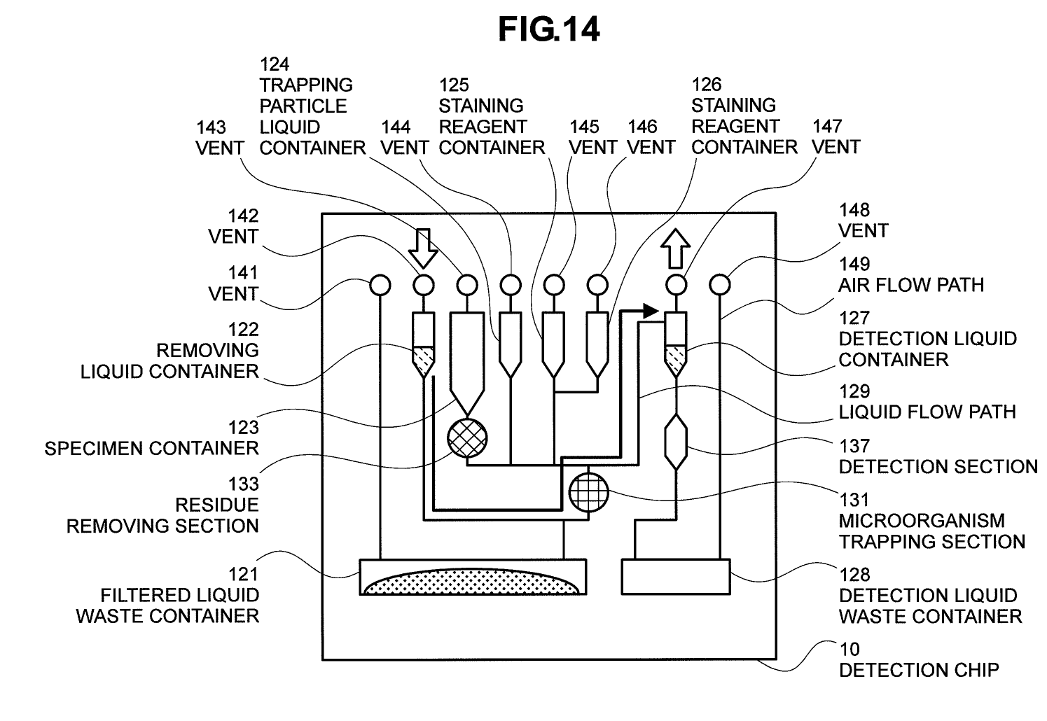

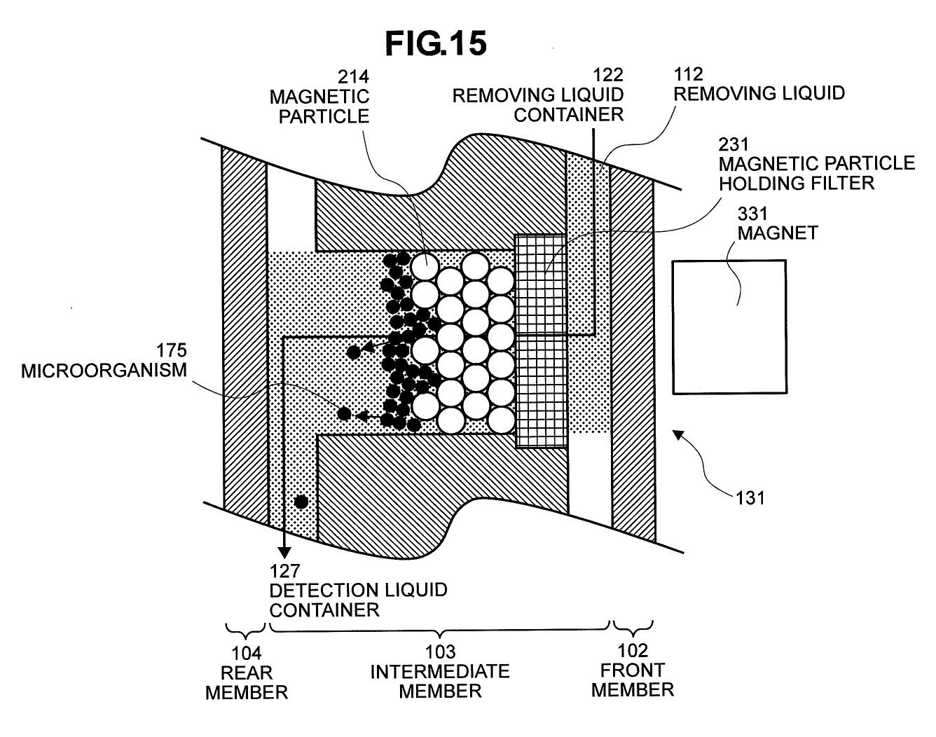

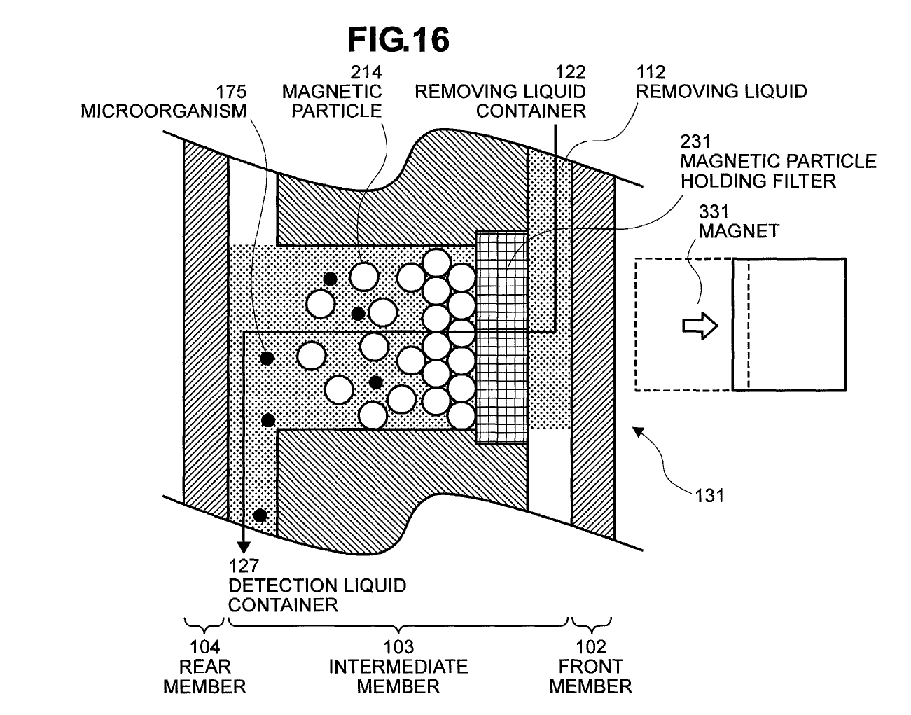

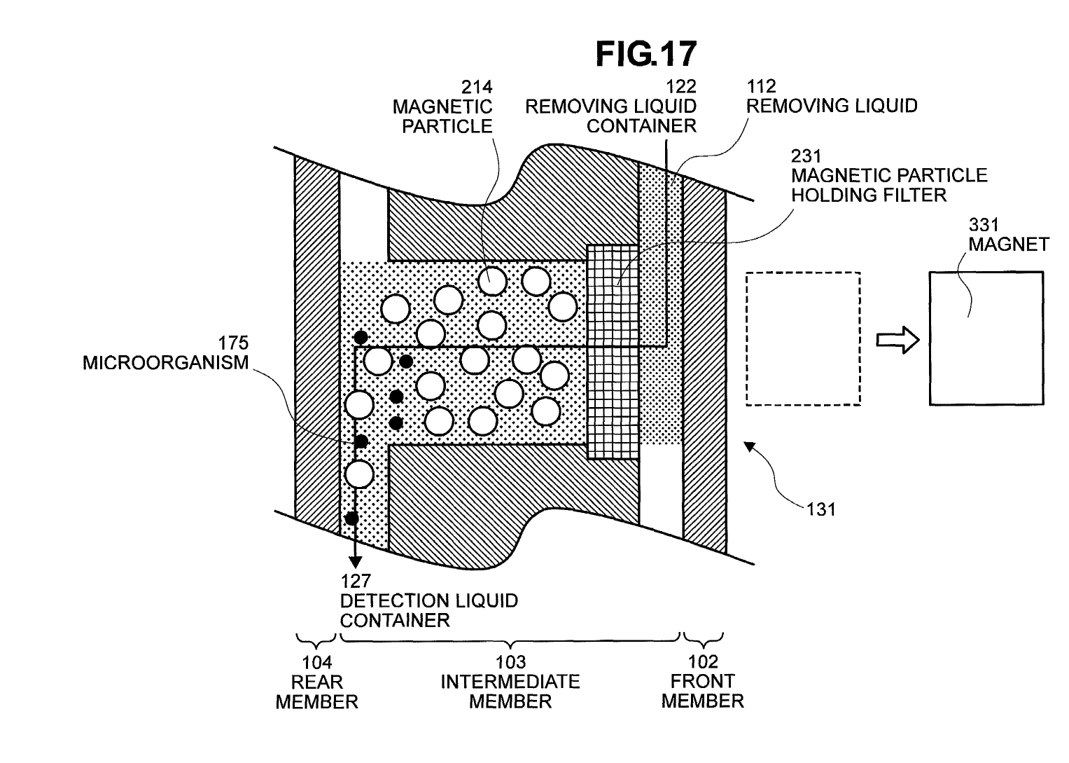

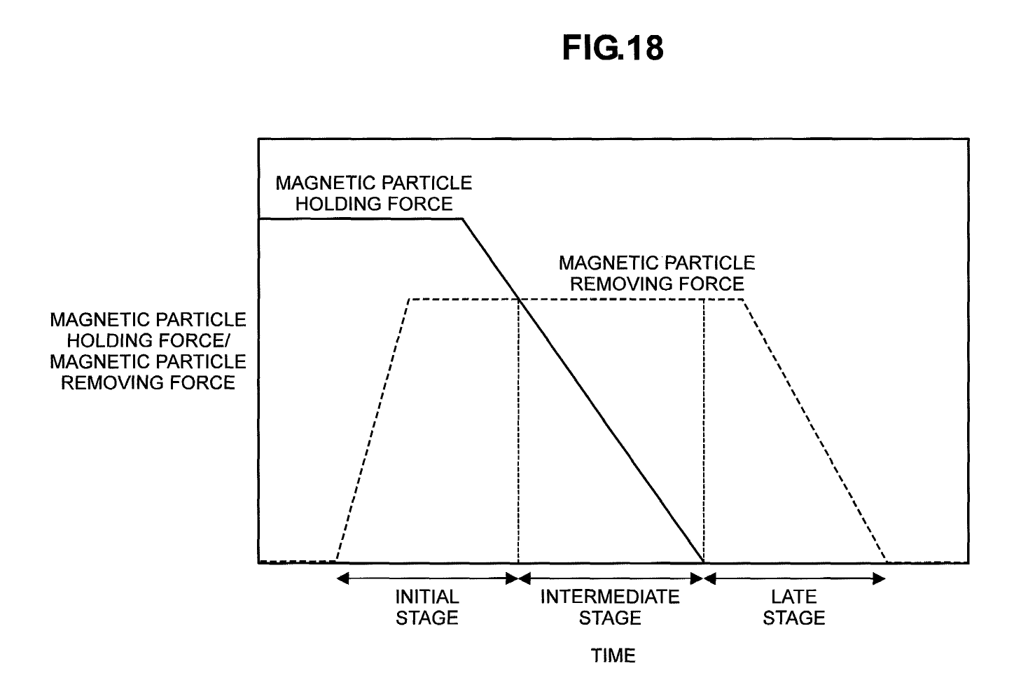

Next, the process for removing the microorganisms 175 will be described in detail with reference to FIGS. 14 to 18. FIG. 14 is a diagram showing the flow of the removing liquid 1221 in the detection chip 10 of FIG. 1. FIG. 15 is a vertical cross-sectional view of the microorganism trapping section 131 in the initial stage of the flow of the removing liquid 1221 in FIG. 14. FIG. 16 is a vertical cross-sectional view of the microorganism trapping section 131 in the intermediate stage of the flow of the removing liquid 1221 in FIG. 14. FIG. 17 is a vertical cross-sectional view of the microorganism trapping section 131 in the late stage of the flow of the removing liquid 1221 in FIG. 14. FIG. 18 is a diagram showing the changes in the magnetic particle holding force and the magnetic particle removing force.

As shown in FIG. 14, the air pressure within the removing liquid container 122 is raised by applying the pressure from the carrier device 20 to the removing liquid container 122 through the vent 142. At the same time, the detection liquid container 127 is opened to the atmosphere through the vent 147. The other vents 141, 143 to 146, and 148 are closed. The removing liquid 1221 flows from the removing liquid container 122 to the detection liquid container 127 through the microorganism trapping section 131, due to the difference between the air pressures in the two containers. At the time of passing through the microorganism trapping section 131, the removing liquid 1221 removes the microorganisms 175 trapped by the filtration filter of the magnetic particles 214 that is formed in the microorganism trapping section 131. The volume of the removing liquid 1221 is made smaller than the volume of the specimen 1231, so that the microorganisms 175 can be removed in a concentrated state. In this embodiment, the liquid flow path 129 has a width of 500 μm and a depth of 500 μm. The amount of the deposited magnetic particles 124 is 25 μL. The volume of the removing liquid 1221 is 1 mL.

Further, the removing liquid 1221 is once held in the detection liquid container 127. Thus, it is possible to remove air bubbles mixed into the removing liquid 1221 passing through the microorganism trapping section 131, from the vent 147. Air bubbles may prevent the detection of the microorganisms 175 in the next detection process, so that it is desirable to remove them as much as possible.

The initial stage of the flow of the removing liquid 1221 is a state where the magnet 331 most approaches to the magnetic particles 214, as well as a state where the magnet 331 becomes separated from the magnetic particles 214. At this time, the magnetic particle holding force of the magnet 331 is larger than the magnetic particle removing force of the removing liquid 1221. Thus, all the magnetic particles 214 are held by the magnetic particle holding filter 231. The magnetic particles 214 do not flow out along with the flow of the removing liquid 1221. Only the microorganisms 175 trapped by the filtration filter formed by deposition of the magnetic particles 214 are gradually removed as shown in FIG. 15.

The intermediate stage of the flow of the removing liquid 1221 is a state where the magnet 331 is further separated from the magnetic particles 214. At this time, the magnetic particle holding force of the magnet 331 is smaller than the magnetic particle removing force of the removing liquid 1221, and the difference between the two forces becomes larger. In the intermediate stage of the flow of the removing liquid 1221, as shown in FIG. 16, a part of the magnetic particles 214 flow out along with the flow of the removing liquid 1221.

The late stage of the flow of the removing liquid 1221 is a state where the magnet 331 is most separated from the magnetic particles 214. At this time, the magnetic particle holding force of the magnet 331 is smaller than the magnetic particle removing force of the removing liquid 1221, and the difference between the two forces is the largest. In the late stage of the flow of the removing liquid 1221, as shown in FIG. 17, all the magnetic particles 214 flow out along with the flow of the removing liquid 1221. Then, the delivery of the removing liquid 1221 is stopped after all the magnetic particles 214 have flowed out of the microorganism trapping section 131.

As described above, when removing the microorganisms 175 from the microorganism trapping section 131 by the removing liquid 1221, the relationship between the holding force by the magnetic force of the magnet 331 and the removing force by the flow of the removing liquid 1221 is adjusted to gradually flow the magnetic particles 214 forming the filtration filter to concentrate the microorganisms 175. In this way, it is possible to surely prevent clogging or other malfunction of the fine flow path in the concentration of the microorganisms 175 in the detection chip 10 which is a disposable chip.

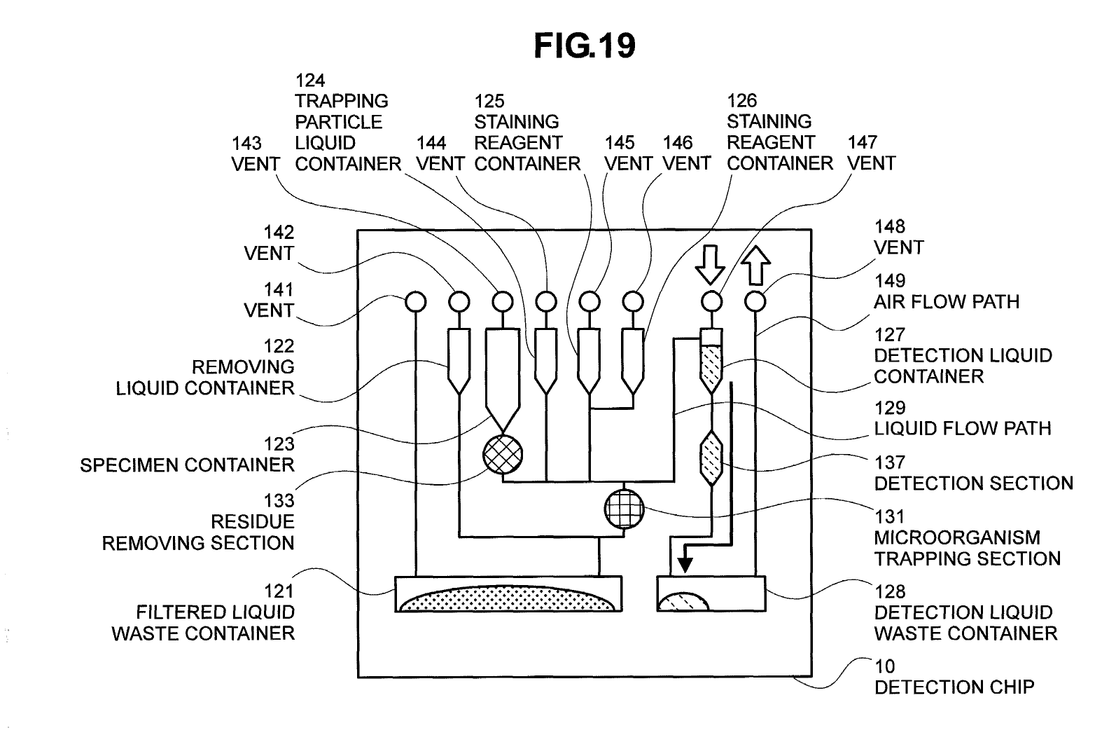

Next, the process for detecting the microorganisms 175 will be described in detail with reference to FIG. 19. FIG. 19 is a diagram showing the flow of the detection liquid 1271 in the detection chip 10 of FIG. 1.

The air pressure within the detection liquid container 127 is raised by applying the pressure from the carrier device 20 to the detection liquid container 127 through the vent 147. At the same time, the detection liquid waste container 128 is opened to the atmosphere through the vent 148. The other vents 141 to 146 are closed. The detection liquid 1271 flows from the detection liquid container 127 to the detection liquid waste container 128 through the detection section 137, due to the difference between the air pressures in the two containers 127 and 128. When the detection liquid 1271 passes through the detection section 137, the microorganisms 175 within the detection liquid 1271 are measured. The measurement of the microorganisms 175 in the detection section 137 is performed by the fluorescent flow cytometry method described above.

As described above, the specimen is moved, concentrated, and stained with the staining reagents, by switching between the sealed state and the open-to-atmosphere state in the specimen container 123, the staining reagent containers 125, 126, and the detection liquid waste container 128, through the vents 141 to 148 of the detection chip 10. In other words, it is possible to consistently perform the removal of residues, concentration of microorganisms, staining of microorganisms, and measurement of the number of living microorganisms, in the single detection chip 10. This reduces the work burden on the inspector as well as the possibility of exposure to the staining reagents, allowing stable measurement results to be obtained without depending on the skill of the inspector. It is also possible to reduce the amount of residues of the used staining reagent, so that the cost necessary for reagents can be reduced.

According to this embodiment, it is possible to realize rapid measurement of the number of microorganisms by the fluorescent flow cytometry method in a single disposable chip, with a pre-process of microorganism concentration incorporated therein. Thus, the number of living microorganisms can be stably measured by a simple operation, while preventing clogging or other malfunction of a fine flow path in the microorganism concentration in the disposable chip.

What is claimed is:

1. A microorganism testing device comprising:
a detection chip having therein a specimen container for holding a specimen containing microorganisms, a trapping particle liquid container for holding a trapping particle liquid containing magnetic particles, a microorganism trapping section for trapping the microorganisms contained in the specimen and a liquid flow path;
a carrier device for applying a carrying force to the specimen held in the specimen container and to the trapping particle liquid held in the trapping particle liquid container;
a controller for controlling the carrier device;
a magnet for holding the magnetic particles in the microorganism trapping section by a magnetic force; and
a detector for detecting the microorganisms flowing in the detection chip,
wherein, in a state where a magnetic force of the magnet acts on the microorganism trapping section, the controller controls the carrier device to flow the trapping particle liquid into the microorganism trapping section to form a filtration filter by trapping and holding a plurality of the magnetic particles in the microorganism trapping section, and in this state, to flow the specimen into the microorganism trapping section so that the microorganisms within the specimen are deposited on one side of the filtration filter.

2. The microorganism testing device according to claim 1,
wherein the detection chip has therein a removing liquid container for holding a removing liquid,
wherein the carrier device applies a carrying force to the removing liquid held in the removing liquid container, and
wherein the microorganism trapping section has a magnetic particle holding filter provided in a portion of the liquid flow path, to form the filtration filter by trapping the magnetic particles on the magnetic particle holding filter.

3. The microorganism testing device according to claim 2,
wherein, in a process for removing the microorganisms deposited in the filtration filter, the magnet changes from a first state where the magnetic particle holding force for holding the magnetic particles is larger than the magnetic particle removing force for removing the magnetic particles from the magnetic particle holding filter, to a second state where the magnetic particle holding force for holding the magnetic particles is smaller than the magnetic particle removing force for removing the magnetic particles from the microorganism trapping section.

4. The microorganism testing device according to claim 3,
wherein the magnetic particle holding force of the magnet for holding the magnetic particles is gradually reduced to allow the magnetic particles to gradually flow out of the magnetic particle holding filter.

5. The microorganism testing device according to claim 4,
wherein the magnet is movably provided on the side opposite to the filtration filter with the magnetic particle holding filter interposed therebetween.

6. The microorganism testing device according to claim 2,
wherein, in the initial stage of a process for removing the microorganisms, the removing liquid is allowed to flow by applying a magnetic force to hold all the magnetic particles on the magnetic particle holding filter,
wherein, in the intermediate stage of the process for removing the microorganisms, the removing liquid is allowed to flow by reducing the magnetic force to a level to hold some of the magnetic particles on the magnetic particle holding filter, and
wherein, in the late stage of the process for removing the microorganisms, the removing liquid is allowed to flow by further reducing the magnetic force to a level to allow all the magnetic particles to flow out of the magnetic particle holding filter.

7. The microorganism testing device according to claim 2,
wherein the detection chip has a multi-layer structure including a front member, an intermediate member, and a rear member, and
wherein the microorganism trapping section has grooves formed on both surfaces of the intermediate member, a through hole through which the grooves communicate with each other, and the magnetic particle holding filter provided in the through hole.

8. The microorganism testing device according to claim 2,
wherein the volume of the removing liquid held in the removing liquid container is smaller than the volume of the specimen held in the specimen container.

9. The microorganism testing device according to claim 2,
wherein the detection chip has therein a staining reagent container and a detection section,
wherein the carrier device applies a carrying force to the staining reagent held in the staining reagent container,
wherein the controller controls the carrier device to flow the staining reagent into the microorganism trapping section to stain the microorganisms deposited in the filtration filter, allowing a detection liquid that contains the removing liquid as well as the stained microorganisms removed from the filtration filter, to flow to the detection section, and
wherein the detector irradiates light onto the detection liquid flowing through the detection section, detects fluorescence or scattering light from the stained microorganisms, and converts the detected light into electrical signals to measure the number of the relevant microorganisms.

10. A chip for testing microorganisms, comprising:
a specimen container for holding a specimen;
a residue removing section for removing residues within the specimen;
a microorganism trapping section for trapping microorganisms within the specimen;
a trapping particle liquid container for holding magnetic particles to form a filtration filter in the microorganism trapping section;
a staining reagent container for holding staining reagent;
a filtered liquid waste container for receiving the specimen, the trapping particle liquid, and the staining reagent that have passed through the microorganism trapping section;
a removing liquid container for holding a removing liquid;
a detection liquid container for holding a detection liquid which is a liquid of the microorganisms removed from the microorganism trapping section;
a detection section for detecting the microorganisms;
a detection liquid waste container for receiving the detection liquid having passed through the detection section;
a liquid flow path for connecting the respective containers, through which the specimen, the trapping particle liquid, the staining reagent, and the removing liquid flow;
a vent for allowing the specimen, the trapping particle liquid, the staining reagent, and the removing liquid that are held in the respective containers, to flow by air pressure; and
an air flow path for connecting the vent and the respective containers,
wherein the microorganism trapping section includes a magnetic particle holding filter to form a filtration filter by depositing a plurality of the magnetic particles, in which a magnetic force is applied to the magnetic particles forming the filtration filter.
